(19) United States
(12) Patent Application Publication
(10) Pub. No.: US 2013/0131440 A1
(43) Pub. Date: May 23, 2013

(54) Orogastric calibration tube with magnets

(71) Applicant: Nick H. Gabriel, Hauppage, NY (US)

(72) Inventor: Nick H. Gabriel, Hauppage, NY (US)

(21) Appl. No.: 13/573,713

(22) Filed: Oct. 3, 2012

Related U.S. Application Data
(60) Provisional application No. 61/542,444, filed on Oct. 3, 2011.

(51) Int. Cl. A61M 29/02; A61F 2/00

(52) U.S. Cl. CPC A61M 29/02; A61F 2/0063
USPC 600/37; 606/192

(57) ABSTRACT

An orogastric calibration tube has a magnet tip attached at a distal end of the flexible tube. The tube also includes a balloon cavity, and means for inflating the balloon cavity.

### CROSS REFERENCE TO RELATED APPLICATIONS

The present application is a non-provisional of U.S. Application Ser. No. 61/542,444, and hereby incorporates herein by reference the disclosures thereof.

### BACKGROUND

1. Technical Field

The present invention relates to surgical tools and, more particularly, to tools used in bariatric surgery.

2. Discussion of Art

Three principal modes of bariatric surgery, related to the present invention, are employed to reduce stomach size and thereby mitigate extreme obesity. These modes are known as “lap band,” “reduction,” or “plication.”

In lap band surgery, an inflatable silicone band is implanted around an upper part of a patient's stomach and is inflated to restrict that part of the stomach, thereby slowing passage of food from the upper part of the stomach into a lower (larger) part of the stomach. The inflatable band can be removed by a follow up surgery, after desired weight loss has been achieved.

By contrast, gastric plication involves suturing the entire stomach to produce a narrowed, banana-like volume extending from the esophagus to the duodenum.

Finally, gastric reduction or gastrectomy involves surgically resecting an excess portion of the stomach, and suturing together the edges of the resection, to provide a smaller stomach volume.

Lap band surgery may be implemented in combination with one of the other two modes of surgery, whereby synergistic reduction of stomach size may be achieved with follow-on reduction of body mass.

Synergistic lap band and plication surgery requires accurate and gentle insertion and positioning of an orogastric calibration tube to guide the plication step. Some calibration tubes may include internal cables for manipulating the tube tip to guide motion of the tube (endoscopic design). Tubes known to the inventor generally are pushed down the esophagus, so that very careful manipulation is required to avert esophageal tears or hang-ups of the tube. The esophageal sphincter is a region of particular concern for hang-ups and for possible injury. An installed lap band may also present risks of injury and hangup during plication. In the combination of a lap band with plication, the plication typically is done after lap band placement, presenting additional risks for the calibration tube to hang up on the lap band.

Accordingly, it is desirable to provide an orogastric calibration tube that can be installed with minimal risk of complication.

### BRIEF DESCRIPTION

According to the present invention, an orogastric calibration tube, for use in gastric plication or reduction surgery, includes a magnet tip, whereby the tube can be pulled from an esophagus into a stomach by manipulation of a second magnet outside the stomach. Thus, a method for gastric plication surgery includes a step of manipulating an orogastric calibration tube by gradual extraluminal adjustment of a magnet adjacent a patient's stomach, whereby the calibration tube is pulled along within the orogastric tract.

These and other objects, features and advantages of the present invention will become apparent in light of the detailed description of the best mode embodiment thereof, as illustrated in the accompanying drawings.

### DRAWINGS

FIG. 1 shows an orogastric calibration tube with blunt or ogive magnet at the tip.

FIGS. 2-3 show steps of inserting the calibration tube of FIG. 1 into a patient's stomach to provide a calibration guide along the lesser curvature of the stomach.

FIG. 4 shows in schematic view steps of a combination gastric band and plication surgery using the calibration tube shown in FIGS. 1-3.

### DETAILED DESCRIPTION OF THE DRAWINGS

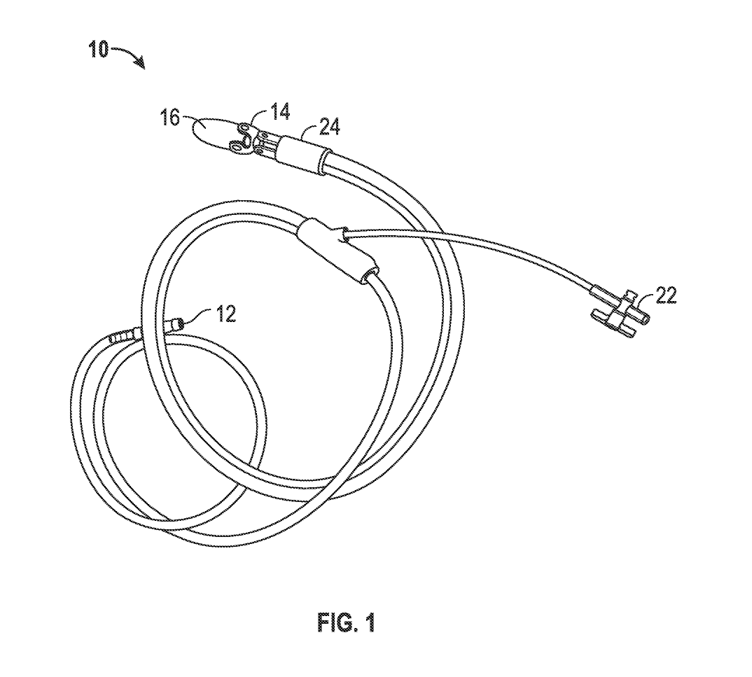

As will be appreciated, the appended drawings show a particular embodiment that is merely illustrative of the inventive concept. In the embodiment shown by FIG. 1, a calibration tube 10 extends from a proximal end 12 to a distal or insertion end 14. The insertion end 14 includes a first magnet 16, which is disposed at the tip of the insertion end. The magnet 16 is preferably blunted or ogive in shape, whereby it can move smoothly downward through a patient's esophagus.

The calibration tube 10 is constructed of a flexible polymer including a semi-rigid Y-fitting 20 that fluidly connects an interior volume of an ancillary tube 22 to an balloon cavity 24 of the calibration tube. The ancillary tube 22 extends from the Y-fitting 20 toward the proximal end 12 of the calibration tube 10, whereby the ancillary tube and Y-fitting can be used as means for injecting saline to inflate the balloon cavity 24 while the calibration tube 10 is in a patient's stomach.

In preferred embodiments, the balloon cavity 24 surrounds the calibration tube 10 and extends along its axis at a portion proximate the distal end 14. Thus, injecting saline into the balloon cavity forms the cavity into a rigid banana shape, adjacent the magnet tip 16, which makes the orogastric calibration tube 10 especially suitable for use in calibrating gastric plication or reduction (gastrectomy). (In other embodiments, rather than extending along an axis of the tube 10, as shown in FIG. 1, the balloon cavity 24 is roughly spherical in shape and is disposed adjacent the magnet 16.)

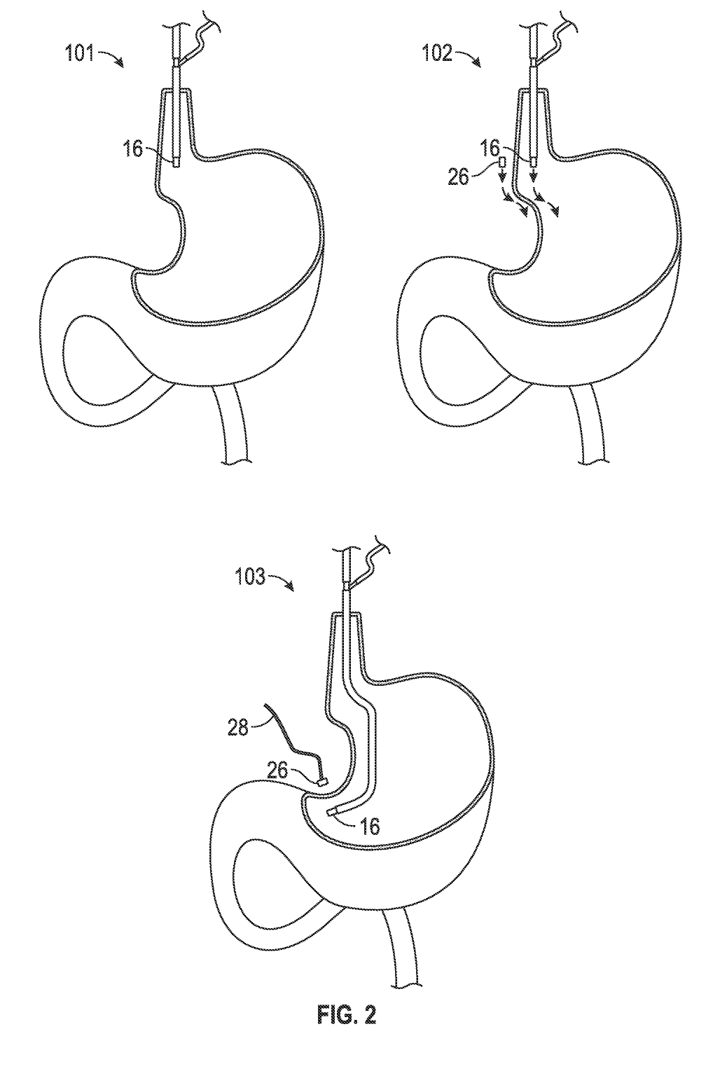

In a method of use 100, as shown in FIG. 2, at step 101 the insertion end 14 is introduced into a patient's orogastric tract by mouth, and is gently urged down the esophagus to near the esophageal sphincter. At or near the sphincter, at step 102 an extraluminal second magnet 26 is introduced to the procedure. The extraluminal magnet 26 engages the first magnet 16 through the stomach wall, and is used to pull the first magnet 16 through the esophageal sphincter and along the lesser curvature of the stomach. Thus, the extraluminal magnet 26 is used to appropriately position the first magnet 16 and the calibration tube 10 within an upper part of the stomach to be plicated or reduced. At step 103 the orogastric calibration tube 10 is positioned along the lesser or inward curvature of the stomach to provide a template to which the upper stomach will be plicated or reduced. At step 103, also, the extraluminal magnet 26 may be held, sutured, or glued in place to hold the first magnet 16 in its appropriate position. The extraluminal magnet 26 remains in position throughout the ensuing procedure, whereby the calibration tube 10 is held in place.

As will be appreciated, the extraluminal magnet 26 may be introduced and manipulated via open technique, or via laparoscopy. In some embodiments, the extraluminal magnet 26 may be tethered on a ribbon or suture 28, whereby loss of the magnet is prevented. Such a ribbon or suture also can ease recovery of the extraluminal magnet at conclusion of an intervention.

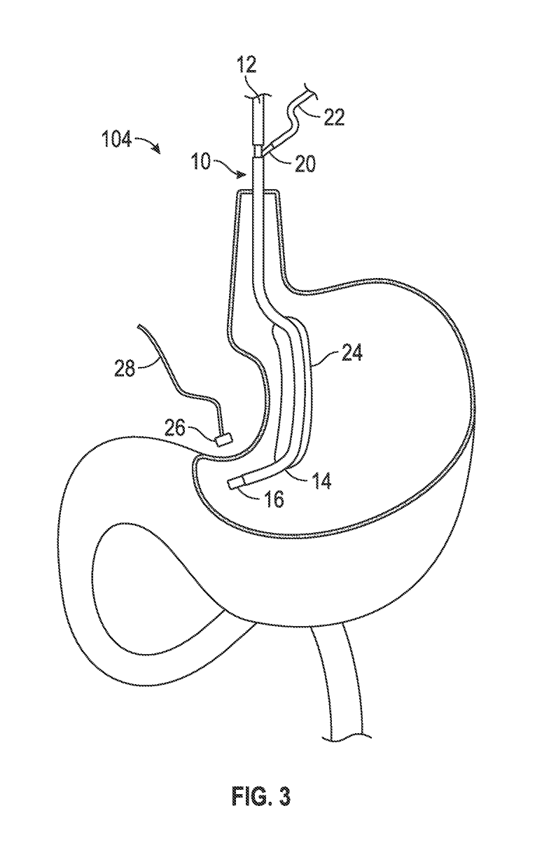

Once the calibration tube 10 is held in place, then at step 104 (as shown in FIG. 3) it can be inflated by injecting saline through the ancillary tube 22 into the balloon cavity 24. Inflation of the calibration tube 10 defines a stomach template to which the patient's stomach will be adjusted by plication or reduction surgery. Thus, it is important that the calibration tube 10 remains held in place by attraction of the magnet tip 16 to the extraluminal magnet 26. In some embodiments, the extraluminal magnet 26 may be sutured or otherwise secured to the stomach wall for fixing the position of the magnet tip 16.

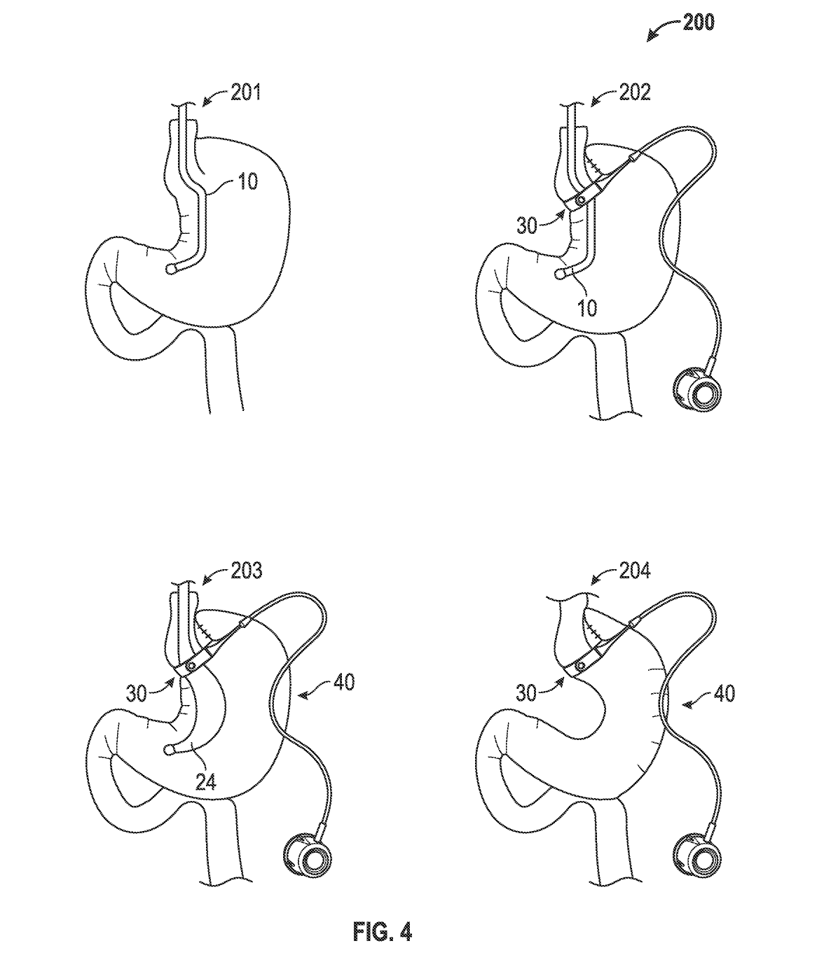

Referring to FIG. 4, the inventive calibration tube 10 can be used as part of an intervention 200 including both plication and lap band surgery. At a first step 201 of such intervention, the calibration tube 10 is inserted according to the method discussed above, although the balloon cavity 24 is not inflated. In other words, positioning of the tube 10 stops at step 103. Once the tube 10 has been positioned, then at step 202 of the intervention 200 a lap band 30 is installed. At step 203, sutures 40 are installed for placating the stomach, and the balloon cavity 24 is inflated. At step 204 the sutures 40 are drawn tight against the balloon cavity 24, whereby the stomach wall 32 is formed to the calibration tube 10 as a guide. The tube 10 then is deflated, and is removed from the stomach transorally. In a possible variant, the tube can be inflated before installing the lap band, thus providing a template for fitting the lap band.

In other embodiments (not shown), one or more magnetized bougies may be used in place of, or in addition to, the intra-gastric magnet tip. (“Bougies” typically are tapered cylinders commonly used to dilate constricted regions of structures such as the esophagus; however, bougies may be of various quasi-cylindrical shapes. In this context, the bougies are used not for dilation but to set a minimum size for plication.) Advantageously, these bougies may be emplaced prior to installation of a lap-band type device, so as to better form the upper stomach. Taking care not to plicate too tightly, the bougies then can be removed via the esophagus.

One advantage of the present invention is that the calibration tube can be positioned more accurately than previously known. A further advantage is that the calibration tube can be positioned with reduced risk of injury to the stomach lining. Yet a further advantage is that the calibration tube can be held in place throughout an ensuing surgical procedure, without continued endoscopic intervention.

Although this invention has been shown and described with respect to the detailed embodiments thereof, it will be understood by those skilled in the art that various changes in form and detail thereof may be made without departing from the spirit and the scope of the invention.

1. An orogastric calibration tube comprising:
a flexible tube having proximal and distal ends, and including a balloon cavity proximate said distal end; and
a magnet tip attached at the distal end of the flexible tube.

2. A bariatric surgical method comprising:
inserting transorally into a patient's orogastric tract an orogastric calibration tube according to claim 1;
manipulating a magnet extraluminal to the orogastric tract, said extraluminal magnet attracting the magnet tip of said orogastric calibration tube, thereby positioning said orogastric calibration tube against the lesser curvature of the patient's stomach; and
inflating the balloon cavity of said orogastric calibration tube,
wherein said orogastric calibration tube forms a template for bariatric surgery.

3. An orogastric calibration tube as claimed in claim 1, further comprising an ancillary tube fluidly communicating with the balloon cavity for inflation thereof.

4. An orogastric calibration tube as claimed in claim 3, wherein the ancillary tube is mounted to the flexible tube by a Y-fitting.

5. An orogastric calibration tube as claimed in claim 1, further comprising means for inflating the balloon cavity.

6. An orogastric calibration tube as claimed in claim 1, wherein the magnet tip is ogive.

7. An orogastric calibration tube as claimed in claim 1, wherein the balloon cavity is configured to form a banana shape upon inflation.

8. A bariatric surgical method as claimed in claim 2, further comprising securing the extraluminal magnet in position, prior to inflating the balloon cavity.

9. A bariatric surgical method as claimed in claim 2, further comprising securing the extraluminal magnet to the stomach wall, prior to inflating the balloon cavity.

10. A bariatric surgical method as claimed in claim 2, further comprising installing a lap band, prior to inflating the balloon cavity.

11. A bariatric surgical method as claimed in claim 2, further comprising installing a lap band, after inflating the balloon cavity; and deflating the balloon cavity, after installing the lap band.

12. A bariatric surgical method comprising:
inserting transorally into a patient's orogastric tract a magnetized bougie;
manipulating a magnet extraluminal to the orogastric tract, said extraluminal magnet attracting said magnetized bougie, and thereby positioning said magnetized bougie against the lesser curvature of the patient's stomach; and
plicating the patient's stomach against the magnetized bougie as a template.

13. A bariatric surgical method as claimed in claim 12, further comprising securing the extraluminal magnet to the stomach wall, prior to plicating the patient's stomach.
